(19) United States
(12) Patent Application Publication
(10) Pub. No.: US 2004/0063218 A1
(43) Pub. Date: Apr. 1, 2004

(54) Immunoassay device and method

(76) Inventors: Frederic Buffiere, des Lavandieres (FR); Christine Betremieux, Chaussee des Bonniers (FR); Jean Alain Chevaleyre, rue Curie (FR); Laetitia Gaillard, Place des Bruyeres (FR); Sandie Menard, rue Paul Fort (FR); Gerard Ovlaque, Chemin de Funquereau (FR); Vinzia Christophe, Le Jardin des Raines (FR)

Correspondence Address: Blakely Sokoloff Taylor & Zafman, 7th Floor, 12400 Wilshire Boulevard, Los Angeles, CA, 90025, US

(21) Appl. No.: 10/433,975

(22) PCT Filed: Dec. 7, 2001
(86) PCT No.: PCT/FR01/03888

(30) Foreign Application Priority Data
Dec. 8, 2000 (FR) 00/16024

(51) Int. Cl. G01N 33/558; C12M 1/34

(52) U.S. Cl. 436/514; 435/287.2

(57) ABSTRACT

The invention concerns a biological assay device using a reaction between an analyte present in a fluid and a reagent capable of forming a complex with said analyte, wherein the analyte is in the form of cellular elements, said device comprising at least a reaction container (1) provided with a reaction zone (2) wherein are introduced the fluid and the reagent, and with an immobilizing zone (3) whereon is fixed a substance (4) capable of specifically binding with the possibly formed complexes, the reaction zone (2) and the immobilizing zone (3) being separated from each other by a material layer (8), said material being capable of shifting from one first state wherein the layer (8) is substantially impermeable to a second state wherein the layer (8) is capable of allowing through the cellular elements, whether complexed or not.

The invention relates to a biological assay device and to its method of use.

It particularly relates to immunoassays based on the reaction between an analyte present in a fluid, especially biological, and a reagent capable of forming a complex with said analyte, in which the analyte and/or the reagent is in the form of formed elements.

It applies typically to searching, in blood or in a blood constituent, for antigens present on the surface of red blood cells by means of known anti-erythrocyte antibodies (type erythrocyte) or to searching for anti-erythrocyte antibodies by means of red blood cells on which specific antigens are present and/or fixed.

Such methods are already known, examples of which are methods in a liquid medium in haemolysis tubes, a particular disadvantage of which is being only slightly sensitive, and microplate methods requiring repeated washing.

So-called solid-phase methods are also known, for example from WO 98/02752, which utilise the reaction between the analyte and the reagent in a container provided with a reaction zone and an immobilising zone in which:

a substance capable of specifically binding with the optionally formed complexes is fixed in the immobilising zone;

the reaction zone and the immobilising zone are separated from one another by a specific medium.

The function of the medium then on the one hand is to isolate the immobilising zone of the reactive medium and on the other hand to allow, under the effect of external forces, selective passage of the cellular elements whether complexed or not from the reaction zone towards the immobilising zone.

This type of method is widely used, as it has the advantage of preventing washing and thus of being able to be performed in a single step, of being very sensitive and being easy to use by requiring reduced handling.

But these methods have disadvantages, including those detailed hereinbelow.

Placing the medium above the immobilising zone is difficult to undertake, precisely and with a flat surface state, especially automatically, due to the viscosity of the medium.

In particular, the medium has to completely cover the substance capable of specifically binding with the optionally formed complexes so that the latter is not inactivated by direct contact with the reactive medium.

In addition, the media proposed in the prior art are not perfectly impermeable to the reactive medium, especially when the latter is being poured in the reaction zone.

This is the reason WO 98/02752 proposes arranging a physical barrier, for example formed by a porous membrane provided with holes, above the medium so as to improve imperviousness between the reaction zone and the immobilising zone.

But the disadvantage of this solution is to complicate fabrication of the immunoassay device by adding an element to the container.

In addition, this method requires providing a form of particular membrane as a function of the form and/or the size of the container.

The object of the invention is thus to remedy all these disadvantages by putting forward an immunoassay device of solid-phase type which is simple to manufacture, while being reliable and wherein the separation medium between the reaction zone and the immobilising zone is impermeable to the reactive medium so as especially to permit the reactive medium to automatically enter the reaction zone.

Furthermore, and once reaction is complete, the separation medium can, simply and reliably, be made capable of allowing the cellular elements through, whether complexed or not, from the reaction zone to the immobilising zone.

To this end, and in accordance with a first aspect, the invention proposes a biological assay device of the type making use of a reaction between an analyte present in a fluid and a reagent capable of forming a complex with said analyte, wherein the analyte and/or the reagent is in the form of formed elements, said device comprising at least one reaction container provided with a reaction zone wherein are introduced the fluid and the reagent, and an immobilising zone on which is fixed a substance capable of specifically binding with the optionally formed complexes, wherein the reaction zone and the immobilising zone are separated from one another by a layer of a material, said material suitable for passing from a first state wherein the layer is substantially impermeable to a second state wherein the layer is capable of allowing the formed elements through, whether complexed or not.

In accordance with a second aspect, the invention proposes an immunoassay process making use of such a device, providing the steps of:

introducing the fluid containing the analyte into the reaction zone;

introducing the reagent into the reaction zone such that the reaction between the analyte and the reagent can take place;

applying the conditions necessary for having the material pass from its first state to its second state;

applying an external force appropriate for allowing the formed elements through, whether complexed or not, from the reaction zone to the immobilising zone via the layer of material in its second state, so as to put the optionally formed complexes in contact with the substance capable of binding with the latter;

visualising optionally fixing the complexes onto the immobilising zone and/or the possible presence of the non-complex formed elements in the collection zone such as to deduce therefrom the positive or negative analysis character.

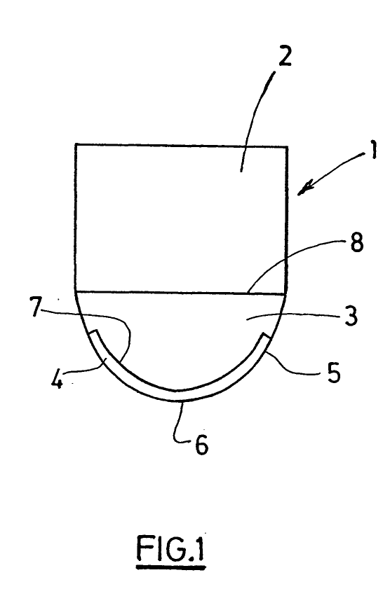

Other objects and advantages of the invention will emerge from the following description with reference to the attached FIG. 1 which illustrates, diagrammatically in section, a container of an immunoassay device according to the present invention.

An immunoassay device comprises at least one reaction container 1, for example made of a stiff plastic material, such as that shown in FIG. 1.

According to an embodiment, the device comprises a plurality of reaction containers 1 so as to produce several identical or different analyses with the same device.

The device comprises for example eight micro-wells 1 of a micro-titration plate of type 96 well having a unit capacity of between 300 and 350 μl, a diameter of around 6 mm and a height of around 8 mm.

In addition, and prior to its being used, the container 1 can be sealed hermetically by a strippable sheet, for example made of special aluminium, so as to avoid possible contamination of its contents.

Each container 1 is intended to allow possible reaction between an analyte present in a fluid, especially biological, and a reagent capable of forming a complex with said analyte.

In the description the terms ‘formed element’ and ‘complex’ refer respectively to the cellular elements of the biological fluid or of the reagent and to the complexes formed by specific links with these elements.

The container 1 receives the reactive medium formed by the biological fluid and the reagent in a first so-called reaction zone 2.

In a particular example the biological fluid is blood or a constituent of blood such as plasma or serum.

The function of each container 1 is also to aid in revealing the in situ positive or negative character of the test, that is, visualising the presence or the absence of complexes.

For this purpose the container 1 is provided with a so-called immobilising zone 3 whereon is fixed a substance 4 capable of specifically binding with the optionally formed complexes.

In the embodiment illustrated in FIG. 1, the container 1 has a U-shape, the opening part forming a reaction zone 2 and the base part forming an immobilising zone 3.

In this embodiment the substance 4 capable of specifically binding with the optionally formed complexes is fixed on substantially the entire involute inner wall 5 of the base part.

In addition, the immobilising zone 3 may comprise a collection zone 6 for the non-complex elements which, in the embodiment shown in FIG. 1, is formed by the lowest central zone of the U.

In other embodiments not shown here other forms of the container 1 can be envisaged, for example V-shaped, or even containers 1 whereof the immobilising zone 3 is convex in shape.

In the type of reaction utilised in the device, the biological fluid and the reagent comprise proteinic elements and/or formed elements.

According to a first use, the aim of the analysis is to reveal the presence of a particular formed element in the biological fluid. The reagent capable of being bound specifically with the desired formed element is then is proteinic form.

A particular example of such analysis is when the analyte is a red blood cell carrying a blood group antigen and the reagent comprises a known antibody capable of binding to this antigen. This analysis can especially determine the group or the phenotype of the red blood cell.

According to a second use, the aim of the analysis is to reveal the presence of a particular proteinic element in the biological fluid. The reagent capable of binding specifically with the desired proteinic element is then in the formed form.

A particular example of such an analyte is when the reagent comprises red blood cells carrying a known blood group antigen and the analyte is an antibody of a serum capable of binding to this antigen. This analysis can especially determine the presence and the nature of an antibody of a type immune prior to a transfusion.

According to a third use, the analyte and the reagent are in the formed form, the aim of the analysis also being to reveal the presence of the analyte in the biological fluid.

A particular example of such analysis is when the reagent comprises lymphocytes expressing a structure able to recognise surface molecules of another cell and the analyte is said other cell.

Described hereinbelow, within the scope of these three particular examples, is an embodiment of the substance 4 capable of specifically binding with the optionally formed complexes.

The chemical and physico-chemical nature of the plastic of the container 1 allows it to be covered in a layer 7 of active molecules of a substance 4 capable of specifically binding with the optionally formed complexes.

The substance 4 is for example formed by antibodies of monoclonal and/or polyclonal origin, especially human anti-immunoglobulin (HAG).

In addition, the substance 4 may comprise antibodies directed against determinants of complementary seric proteins.

The spaces of the inner wall 5 of the bottom of the container 1 which do not comprise the substance 4 can be saturated by saturating agents conventionally used in solid-phase techniques or ELISA (Enzyme Linked Immunosorbent Assay).

This layer 7 which has been applied, for example in the form of a monolayer, in the immobilising zone 3 is capable of recognising any type of human antibodies without particular isotypic specificity and, in the case of anti-complementary antibodies, the fraction C3 of the latter and more particularly of the fractions C3d and C3g carried by the molecule C3.

The substance 4 can be fixed onto the internal wall 5 of the bottom of the container 1 by non-specific means such as passive adsorption, especially antibodies, or by techniques utilising covalent links and allowing structures to be fixed to materials of the plastic type or other.

This monolayer 7, in interacting with the antigens corresponding to it, allows fixing of the complexes optionally formed on the reactive surface.

In the case of a positive reaction, a mat of complexes covering the reactive surface will be observed and, conversely, a point of negativity placed in the collection zone 6 will also be observed.

In an embodiment of the substance 4, a solution of HAG and anti-complementary human antibodies has a concentration of between 1 and 10 μg/ml is prepared in a carbonate buffer of 0.2M pH 9.6.

This solution is distributed in a volume of 75 μl in each well 1 of a micro-plate having a round base of the Maxisorp U8 NUNC type, then the plates are incubated overnight at 4° C.

The micro-wells 1 are then washed by means of a phosphate buffer solution (PBS 2.5 mM pH 7.4) to eliminate all the proteins not absorbed directly into the plastic.

The micro-wells 1 are then treated in an albumin solution at 30 g/l in a PBS buffer at the rate of 100 μl per micro-well.

After incubation of 2 hours at ambient temperature, the micros-wells 1 are washed again in a phosphate buffer.

One of the constraints imposed in the analyses undertaken in the device is that the proteinic elements must remain in the reaction zone 2 so as not to inactivate the substance 4 capable of specifically binding with the optionally formed complexes.

In fact, while the layer 7 HAG is capable of recognising any type of antibodies contained in the biological liquid, any direct contact between the biological liquid and the layer 7 HAG would result in falsely negative analysis.

To have a reliable device, it is thus necessary to isolate the reaction zone 2 from the immobilising zone 3 since the antibodies contained in the biological liquid analysis irrelevant for the analysis in question are capable of returning to compete with the specific antibodies vis-a-vis the layer 7 HAG.

To this end, the invention proposes that the reaction zone 2 and the immobilising zone 3 are separated from one another by a layer 8 of a material, biological in particular, which, in a first state, is substantially impermeable to any fluid.

But, consequent to the reaction, the cellular elements, whether complexed or not, must be able to pass through the layer 8 so that the optionally formed complexes can bind to the substance 4.

For this purpose, the invention proposes that the material forming the layer 8 can pass into a second state wherein it lets the formed elements through, whether complexed or not.

The function of the layer 8 then, in its first state, is to act as a physical barrier relative to the reactive medium and, in its second state, to enable, under the action of external forces, formed elements to be transferred, whether complexed or not.

An exemplary embodiment of the biological material forming the separation layer 8 between the reaction zone 2 and the immobilising zone 3 is described hereinbelow.

In this example, in its first state the biological material is in the form of a solid gel or dense in texture and, in its second state, in the form of a liquid.

After sensitising and saturation of a container 1 as described hereinabove, a biological material formed by a mixture of sodium alginates, bovine albumin, sodium pyrophosphate and calcium chloride is used.

The interactions between these different compounds are not fully known, but all the same it can be ventured that the chains of alginates interact by way of their hydrophobic zones with the albumin molecules and by way of their guluronic acid zones with the calcium ions. The sodium pyrophosphate in turn controls the polymerisation reaction between the calcium and the alginate which, without it, would be too fast to produce a gel with homogeneous reticulation.

The biological material is introduced into the container 1 in liquid form, then the gelling takes place after an incubation period of more than one hour.

This gelling period allows the fluid be well distributed over the substance 4 with a good surface state.

The resulting gel allows distribution of the reagents in the reaction zone 2 at two speeds of the order of 400 μl/sec without causing the reactive medium to leak into the immobilising zone 3.

In the example described in relation to FIG. 1, the immobilising zone 3 is filled with a biological material such that the latter also acts as protection of the substance 4 capable of binding specifically to the optionally formed complexes.

As a variant, it is conceivable that the biological material does not directly cover the substance 4, for example by providing another gel to be arranged in the immobilising zone 3 prior to introduction of the biological material.

In the example described, the passage between the first state and the second is made by a change in phase of the biological material, caused by addition of a specific chemical substance to the reaction zone 2.

As a variant, the biological material further comprises the specific substance chemical capable of having it pass from its first to its second state.

In this variant, at the preferred moment radiation can initiate action of the chemical substance on the material so as to have it pass from its first to its second state.

According to other embodiments, it is conceivable that such passage is caused solely by the action of electromagnetic radiation, for example of the ultrasound or microwave type, and/or by a change in temperature of the biological material.

The specific chemical substance capable of depolymerising the gel described hereinabove is an agent complexing the divalent ions, for example EDTA or sodium citrate, which causes its liquefaction.

Actually, a property of the sodium alginates used is to form, in the presence of divalent ions, a dense array (first state of the biological material). This array is reversible, however, because in the presence of agents complexing the divalent ions, a rearrangement and/or dissociation of the chains of alginates causing liquefaction of the gel (second state of the biological material) is noticed.

The kinetics of this liquefaction is associated with the concentration of the sequestering agent, with the temperature and with optional stirring.

From the moment when liquefaction of the gel is complete, nothing further opposes transfer of the formed elements, whether complexed or not, from the reaction zone 2 to the immobilising zone 3.

This transfer can develop under the action of three forces, used in combination or separately:

gravity, by natural decanting of the formed elements;

adapted centrifugal forces;

magnetic forces where the formed elements have paramagnetic properties.

In the event where this transfer occurs under the action of gravity and/or a centrifugal force, the density of the biological material in the second state can be between the density of the proteinic elements of the reactive medium and that of the formed elements, so that on the one hand the proteinic elements remain in the reaction zone 2 and on the other hand the formed elements, whether complexed or not, pass into the immobilising zone 3.

As a variant, the density of the biological material in its second state can have a gradient along a longitudinal direction.

The separation can also be effected by a difference in physico-chemical affinity, for example by a difference in miscibility, between the biological material on the one hand and the formed elements or the proteinic elements on the other hand.

In all these embodiments, the function of the layer 8 in its second state in terms of its density and/or its composition, is to allow, under the effect of external forces, the passage of the formed elements, whether complexed or not, while preventing passage of the proteinic elements.

In the event of a transfer by means of a magnetic force, this function can also be desirable, in order to prevent transfer of the formed elements whether complexed or not, from carrying the proteinic elements into the immobilising zone 3, especially by a drainage effect.

Preparation do the biological barrier 8 mentioned hereinabove is described hereinbelow.

Preparation of the Various Solutions

Solution of Alginates and Tetra-Soda Pyrophosphate

A solution at 1.2% (weight/volume) of partially hydrolysed sodium alginates (manuronic acid/guluronic acid ratio between 0.8 and 1) and tetra-soda pyrophosphate 15 mM is prepared by dissolution of a dry extract of commercial alginates and sodium pyrophosphate in a buffer of LISS (Low Ionic Strength Solution). The product is vigorously stirred so as to ensure its complete dissolution. The bubbles which might form are eliminated by gentler stirring. This solution is kept at 4° C. Before it is used, the solution will be returned to ambient temperature.

Albumin Solution

A solution of albumin at 150 g/l is prepared by dilution to half in a LISS buffer of a commercial albumin preparation at 300 g/l. This solution is kept at 4° C. Before it is used, the solution will be returned to ambient temperature.

Calcium Chloride Solution

A solution in LISS buffer of calcium chloride with a concentration of 10 mM is used. This solution is kept at 4° C. Before it is used, the solution will be returned to ambient temperature.

EDTA Tetra-Soda Solution

A solution in LISS buffer of tetrasoda tetra-acetic diamine ethylene salt is prepared at a concentration of 100 mM. The pH of this solution is adjusted to 7. This solution is kept at 4° C. Before it is used, the solution will be returned to ambient temperature.

Preparation of the Gel

The extemporaneous mixing before use is carried out according to a precise order.

In the first instance, a volume-to-volume mixture of the solution of alginates and pyrophosphate and the albumin solution is completed. Following gentle stirring for several minutes, a volume of the solution of calcium chloride is added to a volume of this solution. This mixture is homogenised rapidly then distributed in the micro-wells 1 at the rate of 100 μl per well before the commencement of gelling, so as to completely cover the layer HAG 7 (preparation and handling time of such a product must not exceed thirty minutes). Incubation for one hour minimum results in a translucid and resistant gel.

In addition, the gelling step proceeds sufficiently slowly to allow easy industrial manufacturing of the device and the absence of washing allows it to be utilised easily and possibly fully automatically.

The micro-well 1 is then ready for use and must be kept at 4° C.

Depolymerisation of the Gel

Depolymerisation of the gel results from distributing around 50 μl per well 1 of a solution containing 100 mM EDTA (other products complexing the calcium ions can also be used). Complete liquefaction of the gel is attained after incubation of around 15 minutes at a temperature of 20° C.

Preparation and distribution of the layer 8 of biological material are thus done in one step and, the act of mixing the different constituents of this layer 8 according to a precise order and at given concentrations results in a homogeneous gel in all its mass and whereof depolymerisation is perfectly controllable and takes place simply in the axis of the micro-wells 1.

Described hereinbelow are the steps of the process for implementing a ready-for-use device, following removal of the strippable sheet, comprising the steps of:

introducing the fluid containing the analyte into the reaction zone 2;

introducing the reagent into the reaction zone 2 such that the reaction between the analyte and the reagent can take place;

applying the conditions necessary for having the material pass from its first state to its second state;

applying an external force appropriate for allowing the formed elements through, whether complexed or not, from the reaction zone 2 to the immobilising zone 3 via the layer 8 of material in its second state, so as to put the optionally formed complexes in contact with the substance 4 capable of binding with the latter;

visualising optionally fixing the complexes onto the immobilising zone 3 and/or the possible presence of the non-complex formed elements in the collection zone 6 such as to deduce therefrom the positive or negative analysis character.

As a variant, and in the event where the biological material passes from its first state to its second state by the addition of a specific chemical substance, the reagent and the specific chemical substance may be introduced simultaneously such that the reactions on the one hand between the analyte and the reagent and on the other hand between the biological material and the chemical substance happen at the same time.

In another variant, and prior to application of the external force, the reactive mixture present in the reaction zone 2 is subjected to conditions favouring the reaction between the analyte and the reagent, for example incubation so as to increase the speed of the reactions to be made.

In a first embodiment, the external force capable of allowing the formed elements through, whether complexed or not, from the reaction zone 2 to the immobilising zone 3 via the layer 8 of biological material in its second state comprises a centrifugal force.

For this purpose, the intensity, direction and duration of the centrifugal force are adjusted on the one hand to enable transfer of the formed elements, whether complexed or not, from the reaction zone 2 to the immobilising zone 3, as well as optionally to enable the non-complex formed elements to be collected in the collection zone 6 and, on the other hand, to leave the proteinic elements in the reaction zone 2.

In a second embodiment, the external force capable of letting the formed elements through, whether complexed or not, from the reaction zone 2 to the immobilising zone 3 via the layer 8 of biological material in its second state comprises, optionally apart from a centrifugal force, a magnetic force.

The magnetic force is created, for example by a permanent magnet or by an electro-magnet, in a direction substantially longitudinal relative to the container 1.

To this end, the formed elements must either be or must be made sufficiently paramagnetic to migrate from the reaction zone 2 to the immobilising zone 3 under the effect of the magnetic force.

In the event where the formed elements are red blood cells, as reagent or analyte, a method is described hereinbelow to increase their magnetic susceptibility prior to being introduced into the reaction zone 2 without damaging the antigens they carry.

Paramagnetic particles are used which have the essential characteristics of having considerable homogeneity of size (ca. 200 nm), a strong charge of ferromagnetic material (ca. 75% by mass) and a fairly hydrophobic surface state.

These particles are fixed to the surface of the red blood cell for example by way of bovine albumin serum so as to create multiple links of low intensities between the surface of the red blood cell and the particles.

To this end, fixing occurs in two steps, the first consisting of activating the particles using a tacked product and the second being placing these active particles with a suspension of red blood cells, whether treated or not, by proteolytic enzymes.

The resulting red blood cells are attracted by a magnetic field and can thus be used directly or, in a variant, treated by solutions of enzymes generally found in immuno-haematological tests.

First Step: Activation of the Ferro-Magnetic Particles

Particles of type P201 by Ademtech placed with a bovine albumin solution at 0.1% (weight/volume) in PBS buffer of pH 7.2. After incubation of thirty minutes a ambient temperature and with stirring (any magnetic stirring), the particles in suspension are attracted by a magnet and the surfactant depleted of particles is eliminated. The pellet of ‘tacked’ particles can be used directly during the sensitisation phase of the red blood cells.

Second Step: Sensitisation of the Red Blood Cells

The globular suspension placed in LISS buffer is added to the pellet of tacked ferromagnetic particles at an adequate concentration (option of working with cellular suspensions between 0.6 to 10% and previously washed three times, or not, with physiologic water, for instance). After the suspension is perfectly homogenised (verify that there are no more particle aggregates), the latter is incubated for thirty minutes at ambient temperature with gentle though homogeneous stirring (the total reactive volume must be kept moving). The red blood cells are then washed with a PBS buffer of pH 7.4 (two washings by centrifuge, three minutes at 500 g). The pellet of sensitised red blood cells can then be taken up at the concentration for utilising the analyte by a LISS buffer.

In a particular example, the ratio between the quantity of particles used and the quantity of red blood cells is between 600 and 1000 so as to obtain sufficient magnetisation without risking degrading the antigens presents on the surface of the red blood cell.

Using this method thus increases the magnetic susceptibility of the red blood cells without altering the antigen which they carry.

These red blood cells sensitised by the paramagnetic particles then exhibit the double property of being attracted by magnetic field and also having on their surface the blood antigens (group and phenotype). They can then be used as reaction support and transport vector of the antibody couple and can thus pass through the biological material in its second state.

The resulting red blood cells can either be used directly as reagent or as analyte, or undergo treatment by proteolytic enzymes such as papain to perform so-called enzymatic analysis.

As a variant, the antibodies are, prior to being introduced into the reaction zone 2, treated so as to be rendered paramagnetic, for example by means of a method similar to that hereinabove.

Described hereinbelow is a first and a second example of analysis performed using this process.

In the first example, red blood cells, whereof the group and the phenotype are known, are preferred to detect and determine the nature of an antibody of immune type, that is, developed as a result of immunisation during a blood transfusion or pregnancy. The presence of such antibodies can seriously compromise any new blood transfusion not compatible in the system in question and in the event of pregnancy can have serious consequences for the survival of the foetus. This analysis regularly practised is known as Irregular Agglutinine Search (RAI).

To utilise this type of analysis, the reagent comprises red blood cells carrying an antigen of known blood group and the analyte is an antibody capable of binding to this antigen.

The operator then deposits a volume of serum or biological medium to be studied (around 25 μl) in the reaction zone 2 and two volumes of indicative red blood cells solution, for example previously treated with paramagnetic particles. As a variant, this solution may comprise the agent depolymerising the gel.

The operator then deposits the micro-plate or the rod onto an adapted form allowing, if required, incubation at 37° C., then the whole is subjected to a magnetic field and/or a centrifugal force.

Once they make contact with the layer 7 HAG, the antibodies bound specifically to the surface antigens of the red blood cells are going to interact with the antibodies absorbed on the inner wall 5 of the container 1.

Therefore, in the event of a positive reaction an image of a mat of red blood cells, whose homogeneity and dimensions will be functions of the quantity of desired antibodies, will form. If the reaction is negative, a central point of red blood cells will appear in the collection zone 6.

In the second example, it is preferred to determine the group and the phenotype of the red blood cells, and in this case specific probes are used (monoclonal or polyclonal antibodies, vegetable lectins) of certain determinants of the blood groups.

To this end, the analyte is a red blood cell carrying a blood group antigen and the reagent may comprise a known antibody capable of binding to this antigen.

Use of the analysis is thus identical to that mentioned hereinabove with the difference that, in the case of using a magnetic force, it is the red blood cells to be analysed which can be treated so as to increase their magnetic susceptibility.

As a variant, these are antibodies which can be treated such as to be rendered paramagnetic and thus carry along, under the effect of a magnetic force, the red blood cells to which they are bound in the immobilising zone 3.

1. A biological assay device of the type making use of a reaction between an analyte present in a fluid and a reagent capable of forming a complex with said analyte, wherein the analyte and/or the reagent are in the form of formed elements, said device comprising at least a reaction container (1) provided with a reaction zone (2) into which the fluid and the reagent are introduced, and an immobilising zone (3) on which is fixed a substance (4) capable of specifically binding with the optionally formed complexes, characterised in that the reaction zone (2) and the immobilising zone (3) are separated from one another by a layer (8) of a material, said material being capable of passing from a first state wherein the layer (8) is substantially impermeable to a second state wherein the layer (8) is capable of allowing the formed elements through, whether complexed or not.

2. The device as claimed in claim 1, characterised in that the immobilising zone (3) is filled with material such that the latter also serves as protection for the substance (4) capable of specifically binding with the optionally formed complexes.

3. The device as claimed in claim 1 or 2, characterised in that the passage between the first state and the second state is created by changing the phase of the material under the action of electromagnetic radiation, a change in temperature and/or the addition of a specific chemical substance in the reaction zone (2).

4. The device as claimed in claim 3, characterised in that the material, in its first state, is in the form of a solid gel and, in its second state, in the form of a liquid.

5. The device as claimed in claim 4, characterised in that the material is a material of biological nature formed by a mixture of sodium alginates, bovine albumin, sodium pyrophosphate and calcium chloride, so as to form a gel with a homogeneous reticulation, said specific substance chemical being an agent complexing the divalent ions, for example EDTA, for liquefying said gel.

6. The device as claimed in any one of claims 1 to 5, characterised in that the density of the material in the second state is between the density of the proteinic elements of the reactive medium and that of the formed elements.

7. The device as claimed in any one of claims 1 to 6, characterised in that the substance (4) capable of specifically binding with the complexes comprises the antibodies which are fixed, in the form of a monolayer (7), on the inner wall (5) of the base of the container (1).

8. The device as claimed in any one of claims 1 to 7, characterised in that the immobilising zone (3) comprises a collection zone (6) for the non-complex formed elements.

9. The device as claimed in any one of claims 1 to 8, characterised in that it comprises a plurality of reaction containers (1).

10. The device as claimed in claim 9, characterised in that the containers (1) are micro-wells of a micro-titration plate comprising from 8 to 96 wells, said micro-wells being in the shape of a U or a V.

11. The device as claimed in any one of claims 1 to 10, characterised in that, prior to its use, the container (1) is sealed hermetically by a specific aluminium sheet.

12. An immunoassay process making use of a device as claimed in any one of claims 1 to 11, having the steps of:
introducing the fluid containing the analyte into the reaction zone (2);
introducing the reagent into the reaction zone (2) such that the reaction between the analyte and the reagent can take place;
applying the conditions necessary for having the material pass from its first state to its second state;
applying an external force appropriate for allowing the cellular elements through, whether complexed or not, from the reaction zone (2) to the immobilising zone (3) via the layer (8) of material in its second state, so as to put the optionally formed complexes in contact with the substance (4) capable of binding with the latter;
visualising optionally fixing the complexes onto the immobilising zone (3) and/or the possible presence of the non-complex formed elements in the collection zone (6) such as to deduce therefrom the positive or negative analysis character.

13. The process as claimed in claim 12, characterised in that the conditions required to have the material pass from its first state to its second state comprise addition in the reaction zone (2) of a specific chemical substance.

14. The process as claimed in claim 13, characterised in that the reagent and the specific chemical substance are introduced simultaneously.

15. The process as claimed in any one of claims 12 to 14, characterised in that the conditions required to have the material pass from its first state to its second state comprise application of an external physical action, for example the action of electromagnetic radiation and/or a change in temperature.

16. The process as claimed in any one of claims 12 to 15, characterised in that, prior to application of the external force, the reactive mixture present in the reaction zone (2) is subjected to conditions favouring reaction between the analyte and the reagent, for example incubation.

17. The process as claimed in any one of claims 12 to 16, characterised in that the fluid is blood or a constituent of blood such as plasma or serum.

18. The process as claimed in claim 17, characterised in that the reagent comprises red blood cells carrying a known blood group antigen and the analyte is an antibody capable of binding to this antigen, said process especially allowing the presence and nature of an immune type antibody prior to transfusion to be determined.

19. The process as claimed in claim 17, characterised in that the analyte is a red blood cell carrying an antigen of a blood group and the reagent comprises a known antibody capable of binding to this antigen, said process especially allowing the group or the phenotype of the red blood cell to be determined.

20. The process as claimed in claim 18 or 19, characterised in that, prior to being introduced into the reaction zone (2), the red blood cells are treated so that their magnetic susceptibility is increased.

21. The process as claimed in claim 19, characterised in that, prior to being introduced into the reaction zone (2), the antibodies are treated so that they are made paramagnetic.

22. The process as claimed in claim 20 or 21, characterised in that paramagnetic particles are fixed to the surface of the red blood cells and/or to the antibodies, for example by way of molecules of bovine albumin serum.

23. The process as claimed in any one of claims 12 to 22, characterised in that the external force comprises a centrifugal force.

24. The process as claimed in any one of claims 12 to 23, characterised in that the external force comprises a magnetic force.
